(19) United States
(12) Patent Application Publication
(10) Pub. No.: US 2022/0288211 A1
(43) Pub. Date: Sep. 15, 2022

(54) DOSAGE-INDICATING TOPICAL FORMULATION AND METHOD OF USING A DOSAGE-INDICATING TOPICAL FORMULATION

(71) Applicant: Younger Phenotype Inc., Walnut Creek, CA (US)

(72) Inventors: Thomas Henry Nufert, Walnut Creek, CA (US); David Kreimer, Berkeley, CA (US)

(73) Assignee: Younger Phenotype Inc., Walnut Creek, CA (US)

(21) Appl. No.: 17/654,042

(22) Filed: Mar. 8, 2022

Related U.S. Application Data
(60) Provisional application No. 63/158,698, filed on Mar. 9, 2021.

(51) Int. Cl. A61K 47/02; A61K 9/00; A61K 9/06; A61K 45/06; A61K 33/34

(52) U.S. Cl. CPC A61K 47/02; A61K 9/0014; A61K 9/06; A61K 45/06; A61K 33/34

(57) ABSTRACT

A dosage-indicating topical formulation and a method of using said formulation is described. The topical formulation includes one or more bioactive substances and a topical dosage indicator. The topical dosage indicator includes one or more colored copper-containing compounds, and has a color that changes as the topical dosage indicator is absorbed into the skin. The copper-containing compound may include, for example, a copper (II) salt or a copper (I) salt; and a complexation agent containing donor atoms acting as ligands, where the donor atoms are selected from the group consisting of one or more of a nitrogen (N) atom, an oxygen (O) atom, a sulfur (S) atom. The method of using the dosage indicating topical formulation includes repeated rubbing portions of the topical formulation into the skin until the change in color indicates that the skin is saturated with the formulation.

### CROSS-REFERENCE TO RELATED APPLICATIONS

This application claims the benefit of U.S. Provisional Application No. 63/158,698, filed Mar. 9, 2021, the contents of which are hereby incorporated by reference in its entirety.

### BACKGROUND OF THE INVENTION

### Field of the Invention

The present invention generally relates to topical formulations, and more particularly to a dosage indicating topical formulation and method of using a dosage indicating topical formulation.

Discussion of the Background

A variety of topical formulations, such as creams, gels, lotions, cosmetics, cosmeceuticals, sunscreens, balms, ointments, etc., contain various useful substances; useful substances can exert desirable actions at the site of application or can exert desirable systemic effects. The ultimate goal of design of topical formulations is to enable the delivery of useful substances into or onto the skin. The term “useful substances,” as used herein, includes bioactive substances, also referred to herein as “bioactives.” The categories of useful substances may include chemical, biological, nutritional, antimicrobial, antifungal, antiviral, anti-parasite, wound-healing, esthetic, nanostructured materials, and therapeutics. For example, bioactive compounds that can be included into over-the-counter topical formulations include those described in the FDA Monograph entitled “External Analgesic Drug Products for Over-the-Counter Human Use; Tentative Final Monograph” published in the Federal Register, Volume 48, Number 27, 21 CFR Part 348

Routinely, a user apportions a formulation onto the skin liberally, with little sense of how much of the useful substance is being applied. Exceptions involve useful substances that possess color, the disappearance of which from the skin surface can indicate to the user an appropriate application amount.

It is generally assumed that it is safe and effective to apply as much formulation material with each application, as one wishes. Indeed, modern formulations are designed to meet safety requirements when used as directed. However, these directions fail to guide the user to efficiently use the formulation, which is to avoid the application of insufficient or excessive amounts.

When useful substances do not possess an intrinsic color, the efficient use of the formulation can be difficult to achieve without a way to measure or approximate the amount of material applied to the skin. Optimal application of useful substances to the skin can be achieved when the user knows that a sufficient, but not excessive amount of material was applied. For example, the insufficient application of a topical formulation intended to provide pain relief, could result in less pain reduction or a shorter period of being pain-free.

Alternatively, the excessive application results in the waste of product due to a faster rate of consumption of the formulation. Such waste increases customer costs. Excessive application amounts can rub off of the skin and stain clothing and can induce undesirable subjective senses, such as “oily or greasy” skin. In addition, others who touch the user may form an unpleasant impression of “greasy skin.” Finally, although all useful substances obey pharmacological, biochemical and toxicological principles and have established safety profiles, if used in excess, can exert adverse effects. Therefore, it would seem prudent to devise approaches that optimize the dosing of useful substances.

Thus, there is a need in the art for a topical formulation that provides the user with the extent to which the formulation has been absorbed into the user's skin. The topical formulation should effectively provide such an indication to the user and should be biocompatible, and inexpensive to produce.

### BRIEF SUMMARY OF THE INVENTION

The present invention overcomes the disadvantages of prior art topical formulations by the addition of a dosage indicator to the topical formulation. The dosage indicators described herein change color as the topical formulation is absorbed into the skin, where a change from the original color to a different color, or the absence of color, indicates skin saturation. This color change can guide a user to apportion an appropriate amount of the formulation material to achieve more efficient and safe delivery of useful substance(s). The dosage indicator, which is also referred to herein without limitation as a “topical dosage indicator,” or as a “copper color dosage-indicator,” can serve as such a surrogate marker.

One embodiment provides a topical formulation for a skin of an animal, where the topical formulation comprises: one or more bioactive substances; and a topical dosage indicator consisting of one or more colored copper-containing compounds, where the topical dosage indicator has a color that changes as the topical dosage indicator is absorbed into the skin. In certain embodiments, at least one colored copper-containing compound of said one or more colored copper-containing compounds includes: a copper (II) salt or a copper (I) salt; and a complexation agent containing donor atoms acting as ligands, where the donor atoms are selected from the group consisting of one or more of a nitrogen (N) atom, an oxygen (O) atom, a sulfur (S) atom. In certain other embodiments, at least one colored copper-containing compound of said one or more colored copper-containing compounds is a suspension of metal copper or copper oxide particles. In other certain embodiments, at least one colored copper-containing compound of said one or more colored copper-containing compounds is a bioactive substance. In yet other embodiments, one or more bioactive substances is said one or more colored copper-containing compounds. In other embodiments, at least one bioactive substance of said one or more bioactive substances is an analgesic, is an anesthetic, is an anti-inflammatory, is an antifungal, is an antimicrobial, is an antiviral, is an anti-parasitical, is a wound-healing agent, is a nutrient, is a cosmetic agent, has a nutritional activity, or is a combination thereof. In certain embodiments, the copper of the one or more colored copper-containing compounds is between 0.1% to 10% by mass of said topical formulation. In yet other embodiments, the topical formulation further includes: one or more compounds that form a gel, a cream, an ointment, a lotion, or a paste, and one or more compounds that preserve said topical formulation.

Another embodiments provides a method of using a topical formulation which is absorbable into a location of a skin of an animal, where said topical formulation includes one or more bioactive substances and a topical dosage indicator consisting of one or more colored copper-containing compounds, where the topical formulation has initial color which is different than a skin color of the skin at the location, and where the topical formulation includes n topical formulation portions where n>=2. The method includes using a first portion (n=1) of the topical formulation including applying the first portion to the location of the skin, rubbing the first portion into the skin for a first duration of time, where the first duration of time is the time required for the first portion of the topical dosage indicator to be absorbed into the skin sufficient for the color of the skin as seen through the rubbed topical formulation to be the same as the skin color at the location; and using sequential portions of the topical formulation (n=2, 3, . . . ) to the location of the skin including applying an n^{th} portion of the topical formulation to the location of the skin, rubbing the n^{th} portion into the skin for an n^{th} duration of time, where the n^{th} duration of time is the time required for the n^{th} portion of the topical dosage indicator to be absorbed into the skin sufficient for the color of the skin as seen through the rubbed topical formulation to be the same as the skin color at the location, and if the n^{th} duration of time is N times greater than the previous, (n−1)^{th} duration of time, stop applying and rubbing the topical formulation to the skin.

Yet other embodiments provide a combination of: a topical formulation having an initial color, said topical formulation comprising one or more bioactive substances and a topical dosage indicator consisting of one or more colored copper-containing compounds, where a color of the topical dosage indicator changes as the topical dosage indicator is absorbed into a skin of an animal, and where the topical formulation includes n topical formulation portions where n>=2, and instructions for applying the topical formulation to the skin, where the instructions include guidance to: use a first portion (n=1) of the topical formulation to apply the first portion to the location of the skin, rub the first portion into the skin for a first duration of time, where the first duration of time is the time required for the first portion of the topical dosage indicator to be absorbed into the skin sufficient for the color of the skin as seen through the rubbed topical formulation to be the same as the skin color at the location; and use sequential portions of the topical formulation (n=2, 3, . . . ) to the location of the skin to apply an n^{th} portion of the topical formulation to the location of the skin, rub the n^{th} portion into the skin for an n^{th} duration of time, where the n^{th} duration of time is the time required for the n^{th} portion of the topical dosage indicator to be absorbed into the skin sufficient for the color of the skin as seen through the rubbed topical formulation to be the same as the skin color at the location, and if the n^{th} duration of time is N times greater than the previous, (n−1)^{th} duration of time, stop the application and rubbing of the topical formulation to the skin.

These features together with the various ancillary provisions and features which will become apparent to those skilled in the art from the following detailed description.

### DETAILED DESCRIPTION OF THE INVENTION

The present invention includes a dosage indicator that, when added to a topical formulation, is a topical dosage indicator that enables a user to easily observe the saturation of the skin with the topical formulation when sufficient levels of useful substances are applied. Upon massaging or rubbing a topical cream, gel, or other formulation into the skin, the topical dosage indicator undergoes a change in color and also becomes more transparent as the formulation penetrates the skin. The topical dosage indicator thus provides an indication of the saturation of the skin with useful substances in the topical formulation.

In certain embodiments, the topical dosage indicator includes copper complexes. Besides exerting their desirable effect on the area of application or systemically, these complexes can serve as an indicator of saturation of the skin in the course of application of the formulation by disappearance of the color. Thus, in some embodiments, colored copper complexes can serve both as a useful substance and as an indicator of the saturation of the skin.

The topical dosage indicator described herein can display various colors. Thus, for example and without limitation, distinct colors associated with the formation of copper complexes are produced when various salts of copper (II) or copper (I) ions are mixed with various complexation agents.

### Topical Formulations Containing Copper Complexes as a Topical Dosage Indicator

There is a long history of design, manufacturing and use of various formulations. Production of these formulations are based upon well-developed science and technology. A number of manufacturers of formulations also offer formulation services for such products, in addition to manufacturing services. For these manufacturers, and others skilled in the art, inclusion of copper complexes into various application formulations as a topical dosage indicator is straightforward.

In one embodiment, an ointment is designed to include colored copper complexes as a topical dosage indicator. Such an ointment consists of a single-phase in which hydrophobic copper complexes are added and dispersed.

Given the chemical complexity of the composition of formulations containing a copper color dosage-indicator, in which copper complexes are present, it makes little practical sense to define copper complexes by specifying exact molecular species and their structures. Copper complexation and its dynamics are complicated because of dependency upon a number of factors. These include pH, competitive ions and complexation agents, molar ratios of complexation agents to copper ion(s), emulsion composition, etc. Therefore, we use the term “copper complexes” to describe any number of species that are formed upon mixing appropriate formulation components with copper ions. The formulation of a copper complex that possesses suitable color as described herein is within the scope of those skilled in the art.

In another embodiment, a gel is designed to include colored copper complexes as a dosage indicator. Such a gel, consisting of liquids gelled by suitable gelling agents, can readily encompass copper complexes is within the scope of knowledge of those skilled in the art.

In another embodiment, a cream is designed to include colored copper complexes as a dosage indicator. The cream consists of a lipophilic phase and an aqueous phase or phases that form an emulsion, which can encompass copper complexes. The details of forming such emulations can be found, for example and without limitation, in the Chapter “Emulsions and Microemulsions” by Gillian M. Eccleston, Encyclopedia of Pharmaceutical Science and Technology, 4th Edition, CRC Press, ePublished 2013, incorporated herein fully by reference.

In designing formulations containing copper complexes as a topical dosage indicator, those skilled in the art may take into account one or more of the following:
(i) Potential chemical or physical interactions of copper complexes with active and inactive ingredients of the formulation.
(ii) Free copper ions in copper (II)-containing formulations, the product of dissociation of copper sulfate, copper chloride, or other copper(II) salts, shall be minimally present in the formulation, as excessive copper(II) ions can cause skin irritation. Various copper complexation agents, such as salicylate, have been described in the prior art and can be suitable for use as a dosage indicator (see, for example Sorenson (1976) Copper Chelates as Possible Active Forms of the Antiarthritic Agents. Journal of Medicinal Chemistry, 1976, Vol. 19, No. 1, P. 135-147, Sorenson (1978) Copper Complexes—A Unique Class of Anti-Arthritic Drugs. Progress in Medicinal Chemistry, Vol. 15, P. 211-260, Sorenson. Anti-inflammatory and anti-ulcer compounds and process. U.S. Pat. No. 6,306,846 B1, Oct. 23, 2001, Gupta. Topical delivery of trace metals for skin care. U.S. Pat. No. 7,569,558 B2, Aug. 4, 2009, Kang. A pharmaceutical composition comprising a copper chelating tetramine and the use thereof. WO 2017/049529 A1, 30 Mar. 2017, Brown and Osborn. Topical cream for alleviating spider veins. US 2003/0104.043 A1, Jun. 5, 2003, and Dorogi et al. Skin treatment compositions containing copper-pigment complexes. U.S. Pat. No. 8,758,809 B2, Jun. 24, 2014. In addition, aliphatic acids and carboxylic acids that are capable of forming copper complexes, including certain amino acids, can be used for the design of a dosage indicator. To avoid skin irritation with free copper ions, when producing formulations supplemented with a dosage indicator, the molar ratio of copper complexation agents to copper ions should be in excess.
(iii) In certain embodiments of this invention, it is desirable to have an esthetically pleasing ocean blue color of the formulation on the skin prior to rubbing or massaging the formulation into the skin. By means of example, for manufacturing 1 kg of a cream containing copper complexes as dosage indicator described under Example 1 below, 0.1 kg of copper sulfate pentahydrate can be an optimal amount. At this amount per 1 kg of cream, a distinct blue color was achieved. At this amount of copper sulfate pentahydrate per 1 kg of cream, copper (in the form of copper (II)) constituted approximately 2.5% of the cream by mass. A 1.5× to 2× increase in this amount of copper sulfate pentahydrate can result in difficulties forming a stable emulsion. A decrease in the amount of copper sulfate pentahydrate to a level as low as 0.02 kg resulted in a faint color, which made the use of the dosage-indicator problematic. Thus, in formulating creams and other formulations supplemented with a dosage-indicator of this invention, the amount of copper by mass of about 2.5%, similar to that employed in the cream, is closer to optimal. Complexation agents of copper (II) present in a given application formulation can affect the intensity and the hue of color. Therefore, the amount of copper needed for a formulation having a dosage-indicator of this invention can vary in the range from about 5.0% to about 0.1% by mass. In some embodiments, the amount of copper can be as high as 10% by mass.
(iv) In the context of formulation of cosmetics containing a dosage indicator, there is the potential for undesirable color combinations when copper complexes are included in cosmetics.
(v) In designing sunscreens containing a dosage indicator, certain copper complexes will absorb light and can undergo undesirable photochemical reactions.
(vi) In designing various creams and other formulations, one should be aware of the potential occurrence of intermediate colors when, upon rubbing, there is a color change from the original color to the final absence of any color. For example, some formulations initially display the change of color from blue to white, and subsequently, with more rubbing, white changes to clear. The appearance of white color might be due to formation of microbubbles of air while rubbing an emulsion-containing formulation. Regardless of explanations, this feature is useful for better identifying when the saturation of the skin occurs.
(vii) In designing creams and other formulations, one should keep in mind that while many copper-containing compounds possess anti-bacterial and anti-fungal activity, these activities may not be sufficient to protect formulation from microbial growth. The presence of undesirable microorganisms in a topical formulation can cause skin irritation and inflammation. Therefore, despite the presence of anti-microbial copper-containing compounds in a formulation of this invention, it can be desirable to include a conventional preservative or preservatives, such as parabens, propylene glycol, ethyl alcohol, benzalkonium chloride and phenol, into a formulation.

### Examples of Colored Copper-Containing Compounds

Copper complexes most suitable for serving as a color topical dosage indicator of this invention are based upon ligands with donor atoms—nitrogen (N), oxygen (O) and sulfur (S). Various copper complexes with these donor atoms are described in the paper by Krasnovskaya and others, titled “Copper Coordination Compounds as Biologically Active Agents” in the International Journal of Molecular Sciences, 2020 June; 21(11): 3965. Many of these complexes possess color.

For example, copper (II) and copper (I) complexes can be formed using a variety of complexation agents containing an amino, carboxylic or thiol group or groups, or combination thereof. By means of example only, such agents can be amino acids and their derivatives and peptides. As described in the U.S. Pat. No. 5,266,560, and used herein, “the term “amino acid” as used herein means an organic acid containing both a basic amino group (NH₂) and an acidic carboxyl group (COOH); thus, they are amphoteric and exist in aqueous solution as dipolar ions. The term “peptide” as used herein includes any amide derived from two or more such amino acids by combination of the amino group of one acid with the carboxyl group of another. Peptides useful in the practice of the present invention will generally have no more than 6 amino acids. Useful amino acids for the purposes of this invention are the alpha-, beta- and gamma-amino acids. The naturally occurring amino acids that have been established as protein constituents are alpha-amino acids. Many other amino acids occur in the free state in plant or animal tissue. Naturally occurring amino acids are those which are synthesized in nature. Other (non-naturally occurring) amino acids can also be readily synthesized and are useful in the practice of the present invention. Examples of amino acids are: alanine, β-alanine, arginine, cystathionine, cystine, glycine, histidine, homoserine, isoleucine, lanthionine, leucine, lysine, methionine, norleucine, norvaline, ornithine, proline, sarcosine, serine, threonine, thyronine, tyrosine, valine, cysteine, homocysteine, tryptophan, α-aspartic acid, β-aspartic acid, asparagine, α-glutamic acid, β-glutamic acid, glutamine, anthranilic acid, hippuric acid, 3,5-dibromotyrosine, 3,5-diiodotyrosine, hydroxylysine, hydroxyproline, isoleucine, phenylalanine, and thyroxine. The amino acids useful in the present invention can optionally have (in addition to those normally occurring in some of such acids) one or more of the substituents SH, NH₂, OH, COOH, CH₂OH, OCH₃, OC₂H₅, SCH₃, SC₂H₅NH, Cl, Br, F, CCH or CN.”

In certain embodiments of this invention, a derivative of proline can be used as a complexation agent, such as: 3-hydroxy-proline; 4-hydroxy-proline; Δ1-pyrroline-5 carboxylate; Δ1-pyrroline-2 carboxylate; Δ1-pyrroline-3 carboxy-5 carboxylate; Δ1-pyrroline-2 carboxy-4 carboxylate; pyrrole-2 carboxylate; 3,4-hydroxy-proline; and L-glutamyl-semialdehyde.

In various other embodiments of this invention, a variety of colored copper complexes can be formed using various complexation agents within a topical formulation or included into a topical formulation. Many of the complexation agents, including those described above, can impart a multitude of color or color mixtures (hues) for use as color-dosage indicators. For example, complexes in which thiol(s), carboxylate(s) and amine(s), or a combination thereof, produce a variety of colors when complexed with copper, ranging from deep red to dark brown, green to blue. A variety of hues, which can have esthetic utility for commercial products, can be formed depending upon a number of factors.

For instance, the color of a copper dosage-indicator that results from interaction of copper ions with a complexation agent that contains a variety of functional groups will vary appreciably. For instance, amino acid complexes will generally display a blue color, whereas an aromatic or aliphatic complexation agent containing exclusively a carboxylic group will display a green color.

Various hues of blue-green colors will result by producing a dosage indicator that uses different molar ratios of amines or carboxylates to form mixed complexes.

Further variations of color for dosage-indicators can result from using complexation agents that associate with copper in a pH-dependent manner. Complexation agents containing thiol(s), amine(s) and carboxylate(s), or combinations thereof, depending on the pKa of the groups, will provide a range of useful colors.

In yet another example, copper (I) nicotinate complexes possess a deep red color and can be used similarly to copper(II) complexes described above, as a dosage-indicator.

In certain embodiments, it can be desirable to use colored copper complexes that possess therapeutic activity. One of the first examples of such complexes are those described in U.S. Pat. No. 4,221,785, the contents of which are incorporated herein by reference. The complexes that display anti-inflammatory and anti-ulcer activity described therein are “the reaction products of copper salts with:
1. aromatic carboxylic acids or their alkaline earth salts;
2. heterocyclic carboxylic acids or their alkaline earth salts;
3. amino acids or their alkaline earth salts;
4. amines; and
5. suitably substituted steroids.”

Another example of colored copper complexes serving as topical dosage indicator that possess a therapeutic activity are provided in U.S. Pat. No. 5,888,522, the contents of which are incorporated herein by references, and in which peptone digests of various proteins with copper(II) salts were “protecting irritated or damaged skin from further oxidative and biochemical damage and thus permitting natural healing processes to progress, for accelerating the rate of healing of burns and surgical wounds, for stimulating melanogenesis, and for increasing the size of hair follicles and the rate of hair growth.”

Other examples of therapeutically active copper complexes, copper particles and copper oxide particles that can serve as a topical dosage indicator are provided by Gadi Borkow in the paper “Using Copper to Improve the Well-Being of the Skin” published in Curr Chem Biol. 2014 August; 8(2): 89-102. Various copper complexes, copper particles and copper oxide particles and their use in respective therapeutic, cosmetic and biocidal fields are described therein.

### Manufacturing of Copper Complexes

Manufacturing of copper complexes is well described in the art. Numerous examples are provided in the U.S. Pat. No. 4,221,785, the contents of which are incorporated herein by reference.

### The Use of Topical Dosage Indicator

The use of a topical formulation having a topical dosage indicator permits one to consistently administer an appropriate dosage of useful substances, since a user can clearly see when skin saturation is achieved. In certain embodiments, the topical formulation having a topical dosage indicator is used by observing how the color changes during sequential applications of the topical formulation. Thus, for example, a user may sequentially: 1) apply a portion of the topical formulation, and 2) rub the portion into the skin while the disappearance of the color is observed. When the disappearance of the color takes a substantially longer time and more rubbing strokes than in the previous cycle, then the skin is at a saturation point and therefore further addition is superfluous. In certain embodiments, the proper useful substance application occurs after the application of two to five topical formulation portions.

In certain embodiments, a user is provided with guidance for use of a formulation containing a dosage indicator such as an explanation of how to interpret the observed color changes. Such explanation can be provided as a package insert, as a part of the product label, or can be provided in a variety of media formats such as a video, photographs or pictures presented on product-associated websites or any number of digital or other forms.

For optimal use, the guidance should prompt a user to put the formulation material, containing a dosage indicator, on the skin in small portions. Upon rubbing a portion into the skin, the user will see a change in color, while observing the degree of effort necessary to elicit the disappearance of color. The user can correlate the pattern of color disappearance with the effort applied upon adding each consequent small portion.

The number of rubbing strokes that were employed to cause the disappearance or change in color is a good measure for the degree of applied effort. If one rubs each portion into the skin while maintaining a consistent frequency of strokes, the duration of rubbing needed to achieve the disappearance of color can be used as a measure for the degree of applied effort.

The explanation can include further guidance on how to conclude when skin saturation is occurring. Examples of guidance statements could include information such as: “When the color of each subsequent portion becomes increasingly difficult to disappear, this indicates that skin saturation is nearly achieved, and further addition of the formulation material will be wasteful and of no use.”

After completion of the application of a formulation using the above protocol, the skin does not change color and does not appear stained. Upon visual inspection, the surface of the skin at the site of application is virtually indistinguishable from the adjacent skin areas. Upon touch, the application site does not feel excessively greasy, nor does it feel unpleasant.

Further, contact of clothing with the site of application will only minimally stain clothing due to efficient use of the formulation material enabled by this invention. Copper complexes are readily washed out of clothing during a normal laundry cycle or with soap and warm water.

Each time, when a formulation is apportioned in cycles as described above, users achieve consistent results owing to consistent delivery of useful substances. For example, consumers of a topical analgesic cream of this invention containing 1.5% menthol as a useful substance (the active ingredient) and copper complexes as dosage indicator (an inactive ingredient), report consistent and reliable pain relief that typically lasts for several hours. They also report that owing to the consistency of application, they can better plan pain-free periods during their day and night. Naturally, this applies to users, who favorably respond to menthol as an analgesic.

A topical analgesic cream containing copper complexes as dosage indicator described in Example 1 displays, upon rubbing into skin, a change of color from blue to white to clear. The use of the dosage indicator is further illustrated by Examples 2, 3 and 4.

### Example 1. Topical Analgesic Cream Containing Copper Complexes

A topical analgesic cream containing copper complexes as a dosage indicator was designed, as specified by the inventors, and manufactured by an FDA-compliant manufacturer, in accordance with requirements for an OTC topical analgesic drug. One embodiment of this invention encompasses an emulsified cream that contains menthol (1.5%) as an active ingredient. Menthol serves as a useful substance exerting an analgesic effect. Inactive ingredients are: cetearyl alcohol, cupric sulfate (as the source of color for the dosage-indicator of this invention), HCl (for pH adjustment), L-leucine, mineral oil, oleth-20, petrolatum, polysorbates 20 and 60, propylene glycol, PEG-75 lanolin, methyl and propyl parabens, sodium hydroxide (for pH adjustment), and water. Copper(II) and its complexes with various components of the cream provided its characteristic ocean blue color. These copper complexes serve as a copper color dosage-indicator: upon rubbing a portion of the cream into the skin, the color changes from blue to white to clear.

Example 2. Color of the Dosage Indicator Changes on the Skin of a Hand Upon Rubbing

Experimental conditions: Approximately 300 mg of a cream containing copper complexes as color-dosage indicator, was applied onto the skin area ˜2.5 cm×˜3.5 cm of the left hand and gently rubbed into the skin with continuous circular motion of the other hand. Total time of rubbing was about 3 min. Pictures of the left hand (not shown) were taken at denoted time points with a fixed position cellular phone camera. An EcobulbPlus Softwite light bulb was used for illumination. The bulb was positioned 80 cm left and 30 cm above the left hand. The experiment was conducted at room temperature (22° C.).

A control photograph was taken prior to the cream application. As expected, the skin color in the control photograph was natural, with a hint of light brown color characteristic of people of Mediterranean origin. In a subsequent photograph taken immediately upon application of the cream, at the point time 0-minutes, a distinctive blue color is seen at the place application of the cream onto the skin, again as expected. In a photograph taken after 1 minute of rubbing, the blue color begins to turn white. In a photograph taken after 2 minutes of rubbing, only a slightly enhanced glossiness of the skin reveals the area of application. Finally, a photograph taken 3 minutes of rubbing, no trace of color can be observed.

### Example 3. The Use of a Topical Analgesic Cream Containing Copper Complexes as Dosage Indicator on Skin of Varying Color

Volunteers that had minor pains at various parts of their body that responded to commercially available menthol-based OTC topical analgesics were asked to use the topical analgesic cream containing copper complexes as dosage indicator. The volunteers were males and females with skin color varying from white to brown to very dark black. To ascertain whether the color of skin would interfere with the reading of copper color dosage-indicator, the volunteers that were selected had: a very tanned skin (a white male with lower back and hip pain); a non-tanned white male (right knee); a dark-skinned female of middle eastern origin (both knees); a light dark-skinned African-American female (right knee), and a very dark-skinned African-American male (the left arm, thumb joint). They used the cream for several days—from three days to up to 10 days.

As guided by the Directions on the label, all participants applied the cream onto their respective affected area not more than 3 to 4 times daily. Each time, they added a small portion of the cream onto the affected area of the skin and rubbed the cream into the skin. Regardless of the color of the skin, all participants reported that they clearly saw initially the blue color of applied cream. As they rubbed the cream into the skin, the color gradually diminished, turning into white when the cream was present on the skin surface only as a thin layer, and then the color completely vanished. They also reported that, as guided by the Directions, repetition of the application/rubbing cycle was accompanied by the same color change pattern—from blue to white to clear. They also noted, that as described under the Directions, typically, in the first two to three cycles, the change from white to clear required approximately the same number of rubbing strokes and the same time (a minute to two to four minutes), but the third to fifth cycle was characterized by a noticeably slower conversion from white to clear. Again, as per the Directions, no further cycles were performed by the participants.

Typically, the whole application took a few minutes, except when a large area was treated, which took up to 10 minutes. In first cycles, a small amount of cream was rubbed in by about 20 to 40 rubbing strokes (one back and one forth movement of the rubbing hand constitutes one stroke). For the very last cycle, 40 to 80 strokes were typically required.

All participants reported consistent pain relief for a typical duration of several hours. The duration of the pain-free period was approximately the same for each participant, but varied from about 2 to 4 hours to up to 24 hours among the group.

### Example 4. Dosage Indicator Changes in the Course of Application Onto a Restricted Area of Skin

Experimental Objectives: The first objective of this study was to explore whether changes in the color of the dosage-indicator is an intrinsic property of the indicator or is due to spreading of the cream on the skin as it is rubbed in. In the above studies, a blue cream containing copper complexes as dosage indicator was applied on an unrestricted area of the skin and could spread substantially. Thus, while rubbing the cream into the skin, users may have distributed the cream over a large area, which might have contributed to the observed color changes. To demonstrate that the change of color is an intrinsic property of the copper color dosage-indicator, in this study we restricted the application area.

The second objective of this semi-quantitative analysis was to illustrate how changes in the dosage indicator can guide a user in decision making as to whether to enter into a new application/rubbing cycle or to stop applying the cream.

Experimental conditions: The area of application was constrained by four pieces of tape placed on the skin of the left arm to form a restricted area. The area was approximately 1.4 cm×1.4 cm (˜2 cm2). Small portions of the cream (30 mg each) were sequentially applied onto the area and gently rubbed into the skin in three application/rubbing cycles. Rubbing was performed by continuous back and forth movement of the tip of the index finger of the right hand. All tip movements were performed carefully to avoid “spillage” of the cream over the tape. The number of strokes were counted (one back-and-forth movement was counted as one stroke). Photographs of the left hand were taken with a fixed position cellular phone camera at denoted number of strokes. An EcobulbPlus Softwite light bulb was used for illumination. The bulb was positioned 80 cm left and 30 cm above the left hand. The experiment was conducted at room temperature (22° C.).

The photographs were semi-quantitatively analyzed by a biophysicist with expertise in the quantitation of biological images. An evaluation of the area of application was performed based upon the following colors and intensities:

Skin color: “+++”—the color of untreated skin; “++”—clearly seen skin, but partially obscured by overlaying cream; “+”—skin that is barely seen; and “−”—the skin that virtually cannot be seen.

Blue Color: “+++”—intense blue color; “++”—clearly seen blue color, but partially obscured by white and/or skin colors; “+”—faint blue color; and “−”—virtually no blue color.

White Color: “+++”—intense white color; “++”—clearly seen white color, but partially obscured by blue and/or skin colors; “+”—faint white color; and “−”—virtually no white color.

Results: The dosage indicator reliably changed color from blue to white to clear in three cycles of application/rubbing of small, equal, portions of the blue cream containing copper complexes into the skin. The change of color from white to clear was consistently more difficult to achieve from cycle to cycle, indicating that skin saturation with the cream was occurring. The increase of almost four times in the number of strokes needed to achieve the change from white to clear skin color in Cycle II vs. Cycle I indicated that the treated skin area was almost saturated at the end of Cycle II. This signals the user to not apply additional cream for the third cycle. Indeed, it was difficult to complete Cycle III. It took many more strokes (850 strokes in Cycle III vs. 360 in Cycle II) to complete Cycle III to reach the stage of clear skin. This is more than seven times the number of strokes in Cycle I! Thus, the dosage indicator can guide the user to apportion the cream to obtain an appropriate level of skin saturation. The dosage indicator helps avoid waste and costs and excessive use of the cream and unnecessary exposure to the active ingredient.

The semi-quantitative aspect of these results is summarized in Table I below:

TABLE I
Changes of Color in the Cycles of Application/Rubbing

| | | | | | Number of | |
|---|---|---|---|---|---|---|
| Cycle #/ | Stage of | Color/Intensity | | | strokes to | |
| Total | the | | | Clear | change the | |
| Strokes | Cycle | Blue | White | Skin | Stage | Notes |
| Cycle I/ | blue | +++ | − | − | 0 | Change from white to clear |
| 120 | white | − | +++ | − | 90 | required only 30 strokes |
| | clear | − | + | ++ | 30 | |
| Cycle II/ | blue | +++ | − | − | 0 | Almost four times (110 strokes) |
| 360 | white | + | ++ | − | 250 | more effort to get from white to |
| | clear | − | − | ++ | 110 | clear - a clear-cut signal to stop |
| Cycle III/ | blue | ++ | − | + | 0 | Difficult to rub in the last portion; |
| 850 | white | − | ++ | − | 700 | total # of strokes were more than |
| | clear | − | − | ++ | 150 | seven times that of Cycle I. |

### Example 4. The Description Provided on a Label of How to Use a Therapeutic Topical Analgesic Cream Containing Copper Complexes as a Dosage Indicator

The following are directions that were provided for the use topical formulations having copper complexes as a dosage indicator:

Directions: Use only as directed, adults and children 12 years of age and older; Apply to the affected area not more than 3 to 4 times daily. Add a small portion, rub, and see as you rub how the dosage-indicator color changes from blue to white to clear. Repeat until white is slow to disappear.

### Example 5. The Description Provided on a Website of How to Use a Therapeutic Topical Analgesic Cream Containing Copper Complexes as a Dosage Indicator

The following are directions that were provided on a website for the use topical formulations having copper complexes as a dosage indicator:

Directions: Use only as directed, adults and children 12 years of age and older: Apply to the affected area not more than 3 to 4 times daily. Typical application takes just a few minutes: add a small portion, rub, and see as you rub how the dosage-indicator color changes from blue to white to clear. Repeat “add, rub and see™” until white is slow to disappear.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment. Furthermore, the particular features, structures or characteristics may be combined in any suitable manner, as would be apparent to one of ordinary skill in the art from this disclosure, in one or more embodiments.

Similarly, it should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

We claim:

1. A topical formulation for a skin of an animal, said topical formulation comprising:
one or more bioactive substances; and
a topical dosage indicator consisting of one or more colored copper-containing compounds, where the topical dosage indicator has a color that changes as the topical dosage indicator is absorbed into the skin.

2. The topical formulation of claim 1, wherein at least one colored copper-containing compound of said one or more colored copper-containing compounds includes:
a copper (II) salt or a copper (I) salt; and
a complexation agent containing donor atoms acting as ligands, where the donor atoms are selected from the group consisting of one or more of a nitrogen (N) atom, an oxygen (O) atom, a sulfur (S) atom.

3. The topical formulation of claim 1, wherein at least one colored copper-containing compound of said one or more colored copper-containing compounds is a suspension of metal copper or copper oxide particles.

4. The topical formulation of claim 1, wherein at least one colored copper-containing compound of said one or more colored copper-containing compounds is a bioactive substance.

5. The topical formulation of claim 1, wherein said one or more bioactive substance is said one or more colored copper-containing compounds.

6. The topical formulation of claim 1, where at least one bioactive substance of said one or more bioactive substances is an analgesic, is an anesthetic, is an anti-inflammatory, is an antifungal, is an antimicrobial, is an antiviral, is an anti-parasitical, is a wound-healing agent, is a nutrient, is a cosmetic agent, has a nutritional activity, or is a combination thereof.

7. The topical formulation of claim 1, where said topical formulation includes: menthol, a copper (II) salt, leucine, one or more compounds that form an emulsion, and one or more compounds that preserve the topical formulation.

8. The topical formulation of claim 1, where said copper of the one or more colored copper-containing compounds is between 0.1% to 10% by mass of said topical formulation.

9. The topical formulation of claim 1, where said topical formulation further includes:
one or more compounds that form a gel, a cream, an ointment, a lotion, or a paste, and
one or more compounds that preserve said topical formulation.

10. A method of using a topical formulation which is absorbable into a location of a skin of an animal, where said topical formulation includes one or more bioactive substances and a topical dosage indicator consisting of one or more colored copper-containing compounds, where the topical formulation has initial color which is different than a skin color of the skin at the location, and where the topical formulation includes n topical formulation portions where n>=2, said method comprising:
using a first portion (n=1) of the topical formulation including
applying the first portion to the location of the skin,
rubbing the first portion into the skin for a first duration of time, where the first duration of time is the time required for the first portion of the topical dosage indicator to be absorbed into the skin sufficient for the color of the skin as seen through the rubbed topical formulation to be the same as the skin color at the location; and
using sequential portions of the topical formulation (n=2, 3, . . . ) to the location of the skin including
applying an n^{th} portion of the topical formulation to the location of the skin,
rubbing the n^{th} portion into the skin for an n^{th} duration of time, where the n^{th} duration of time is the time required for the n^{th} portion of the topical dosage indicator to be absorbed into the skin sufficient for the color of the skin as seen through the rubbed topical formulation to be the same as the skin color at the location, and
if the n^{th} duration of time is N times greater than the previous, (n−1)^{th} duration of time, stop applying and rubbing the topical formulation to the skin.

11. The method of claim 10, where N is greater than or equal to 2, is greater than or equal to 3, is greater than or equal to 4, or is greater than or equal to 5.

12. The method of claim 10, wherein at least one colored copper-containing compound of said one or more colored copper-containing compounds includes:
a copper (II) salt or a copper (I) salt; and
a complexation agent containing donor atoms acting as ligands, where the donor atoms are selected from the group consisting of one or more of a nitrogen (N) atom, an oxygen (O) atom, a sulfur (S) atom.

13. The method of claim 10, wherein at least one colored copper-containing compound of said one or more colored copper-containing compounds is a bioactive substance.

14. The method of claim 10, wherein said one or more bioactive substance is said one or more colored copper-containing compounds.

15. The method of claim 10, where at least one bioactive substance of said one or more bioactive substances is an analgesic, is an anesthetic, is an anti-inflammatory, is an antifungal, is an antimicrobial, is an antiviral, is an anti-parasitical, is a wound-healing agent, is a nutrient, is a cosmetic agent, has a nutritional activity, or is a combination thereof.

16. The method of claim 10, where said topical formulation includes: menthol, a copper (II) salt, leucine, one or more compounds that form an emulsion, and one or more compounds that preserve the formulation.

17. The method of claim 10, where said copper of the one or more colored copper-containing compounds is between 0.1% to 10% by mass of said topical formulation.

18. The method of claim 10, where said topical formulation further includes:
one or more compounds that form a gel, a cream, an ointment, a lotion, or a paste, and
one or more compounds that preserve said topical formulation.

20. The method of claim 10, where said animal is a human

21. The method of claim 10, where said animal is a non-human animal.

22. A combination of:
a topical formulation having an initial color, said topical formulation comprising
one or more bioactive substances and
a topical dosage indicator consisting of one or more colored copper-containing compounds, where a color of the topical dosage indicator changes as the topical dosage indicator is absorbed into a skin of an animal, and where the topical formulation includes n topical formulation portions where n>=2; and
instructions for applying the topical formulation to the skin, where the instructions include guidance to
use a first portion (n=1) of the topical formulation to
apply the first portion to the location of the skin,
rub the first portion into the skin for a first duration of time, where the first duration of time is the time required for the first portion of the topical dosage indicator to be absorbed into the skin sufficient for the color of the skin as seen through the rubbed topical formulation to be the same as the skin color at the location; and
use sequential portions of the topical formulation (n=2, 3, . . . ) to the location of the skin to
apply an n^{th} portion of the topical formulation to the location of the skin,
rub the n^{th} portion into the skin for an n^{th} duration of time, where the n^{th} duration of time is the time required for the n^{th} portion of the topical dosage indicator to be absorbed into the skin sufficient for the color of the skin as seen through the rubbed topical formulation to be the same as the skin color at the location, and
if the n^{th} duration of time is N times greater than the previous, (n−1)^{th} duration of time, stop the application and rubbing of the topical formulation to the skin.

23. The combination of claim 22, where N is greater than or equal to 2, is greater than or equal to 3, is greater than or equal to 4, or is greater than or equal to 5.

24. The combination of claim 22, where the topical formulation includes a plurality of topical formulation portions, and where said instructions include guidance to sequentially apply and rub two or more topical formulation portions of the plurality of topical formulation portions into the location of the skin.

25. The combination of claim 22, wherein at least one colored copper-containing compound of said one or more colored copper-containing compounds is a bioactive substance.

26. The combination of claim 22, where at least one bioactive substance of said one or more bioactive substances is an analgesic, is an anesthetic, is an anti-inflammatory, is an antifungal, is an antimicrobial, is an antiviral, is an anti-parasitical, is a wound-healing agent, is a nutrient, is a cosmetic agent, has a nutritional activity, or is a combination thereof.

27. The combination of claim 22, wherein said one or more bioactive substance is said one or more colored copper-containing compounds.
